(19) United States
(12) Patent Application Publication
(10) Pub. No.: US 2023/0348978 A1
(43) Pub. Date: Nov. 2, 2023

(54) TREATMENT OF ALZHEIMER'S DISEASE

(71) Applicant: SELONTERRA, INC., San Mateo, CA (US)

(72) Inventors: Anne URFER-BUCHWALDER, Belmont, CA (US); Roman Urfer, Belmont, CA (US)

(73) Assignee: SELONTERRA, INC., San Mateo, CA (US)

(21) Appl. No.: 17/791,240

(22) PCT Filed: Jan. 7, 2021
(86) PCT No.: PCT/US21/12553

§ 371 Date: Jul. 7, 2022

Related U.S. Application Data
(60) Provisional application No. 62/957,999, filed on Jan. 7, 2020.

(51) Int. Cl. C12Q 1/6883

(52) U.S. Cl. CPC C12Q 1/6883; C12Q 2600/158; C12Q 2600/106; C12Q 2600/136; C12Q 2600/118

(57) ABSTRACT

The present invention provides compositions and methods using certain GPR4-regulated genes and expression products thereof (namely YAP1, CTGF, CCND3, BDNF, VCL, ITGB3) for diagnosis, treatment and prevention of Alzheimer's disease and Mild Cognitive Impairment. The present invention also relates to a method of identifying therapeutic agents to treat and diagnose Alzheimer's disease or Mild Cognitive Impairment based on the differential expression of GPR4-regulated genes.

### CLAIM OF PRIORITY

This application claims priority of U.S. provisional application 62/957,999 filed on Jan. 7, 2020.

### FIELD OF THE INVENTION

The present invention relates to a method of preventing and treating Alzheimer's disease and/or neurological disorders such as but not limited to Mild Cognitive Impairment based on modulating GPR4 and/or one or more GPR4-regulated genes, or their expression products.

### BACKGROUND OF THE INVENTION

Alzheimer's disease (AD) affects tens of millions of people worldwide and its prevalence continues to rise. Unfortunately, currently there are no reliable and effective methods for diagnosis, treatment or prevention of AD. It is believed that AD is caused by a combination of environment, lifestyle, medical condition and genetic factors. AD often goes unrecognized in its early stages where some treatments might be most effective. Current FDA approved Alzheimer's drugs have significant side effects and only modest effects on improving the patient's daily functioning but do not slow down the disease process or treat the underlying pathology.

Molecular root causes of Alzheimer's disease: Most of AD research has focused on the accumulation of amyloid plaques and neurofibrillary tangles of hyperphosphorylated microtubule associated protein Tau (MAPT). Synaptic dysfunction, impaired synaptic plasticity and dendritic loss are also observedⁱ. Selective loss of GLUR2, an AMPAR (α-Amino-3-hydroxy-5-methyl-4-isoxazolepropionic acid receptors) subunit occurs even before plaque formationⁱⁱ. Research has also suggested AD involves dysfunction in neuronal cell cycle regulation and cell cycle reentryⁱⁱⁱ. Moreover, expression of neurofilament light is markedly decreased in AD patients compared to controls^{iv}. A 25% reduction of SorLA (SorL1/Sortilin-Related Receptor) in cortex and hippocampus has also been noted in autopsy material and cerebrospinal fluid of AD patients^{v}.

APOE4—The Dominant Genetic Factor in AD. One tantalizing clue towards possible treatments for AD comes from genetic research which, in the early 1990s, revealed that people carrying a specific allele of the apolipoprotein E (APOE) gene, namely the APOE4 allele, are at substantially increased risk of developing AD. APOE3 is the normal isoform for all known functions of APOE. The APOE4 variant harbors a G at position 19:44908684 on chromosome 19 in the Genome Reference Consortium Human Build 38 patch release 2/GRCh38.p2 (single nucleotide polymorphism rs429358). Clinical and epidemiological data have indicated that over 60% of AD patients are APOE4 carriers^{vi} with penetrance of APOE4 estimated to be at 60-70% depending on the population and the study. APOE4 is associated with an earlier age of onset with age 68 as mean age of clinical onset for APOE4 homozygotes versus 84 years of mean age of clinical onset for subjects not carrying the APOE4 allele^{vii}. Homozygous neurons carrying the APOE4 genotype on both chromosomes 19 are herein designated as E4E4 neurons. Homozygous neurons carrying the APOE3 genotype on both chromosomes 19 are herein designated as E3E3 neurons.

Subjects are predisposed to develop AD or Mild Cognitive Impairment by their genetic background. Subjects carrying the APOE4 allele have a substantially increased risk of developing AD or Mild Cognitive Impairment. Predisposed subjects may be homozygous or heterozygous for the APOE4 allele. Further, subjects carrying AD familial variants including variants in amyloid precursor protein (APP), presenilin-1 (PSEN1) or presenilin-2 (PSEN2) have a substantially increased risk of developing AD or Mild Cognitive Impairment.

Enhancers exert their regulatory function through binding of cell-type specific transcription factors. Surprisingly and unexpectedly, using the JASPAR CORE database of experimentally defined transcription factor binding sites for eukaryotes, it was found that the APOE4 variant creates a binding motif for the transcription factor NRF1 (nuclear respiratory factor 1)^{viii}. The APOE4 variant changes a non-consensus A nucleotide with 0 appearance in the nucleotide frequency matrix of the NRF1 consensus sequence into a highly conserved, consensus matching G nucleotide. The NRF1 protein sequence is deposited in UniProtKB as “NRF1_HUMAN” with the accession number Q16656-1. NRF1 is a homodimeric transcription factor which mediates the expression of key metabolic and mitochondrial genes playing an important role in the coupling between energy consumption, energy generation, and neuronal activity^{ix}.

Transcription factors binding to enhancers results in the stimulation or alternatively repression of gene transcription. Enhancers can affect the transcription of genes located in cis as far as 2 Mb away on the same chromosome. Enhancers contain the same regulatory elements that are found at the promoter of the genes they regulate. Contacts between distal enhancers and promoters is set-up by the specific recruitment of“looping” factors. In neurons carrying the APOE4 variant, the trigger specific transcription factor NRF1 induces the formation of a secondary chromatin loop affecting the transcription of several genes in its vicinity. The finding revealed that the APOE4 allele is causing transcription effects in brain cells, altering the transcription of a range of genes, including the expression of GPR4 (G protein coupled receptor 4; UniProtKB as “GPR4_HUMAN” with the accession number P46093)^{x}. By contrast, APOE3 does not create a high scoring NRF1 binding site and thus does not typically influence transcriptional regulation of the genes nearby. Further details of the discovery linking the APOE4 genetic locus to NRF1 activity on genes in the vicinity of APOE4 are set out in PCT patent application WO2018/112446.

### SUMMARY OF THE INVENTION

The present invention provides methods of treatment of Alzheimer's Disease or Mild Cognitive Impairment by modulating the activity of GPR4. Modulation of GPR4 activity provides the advantage of modulating genes regulated or influenced by GPR4. Methods of screening for therapeutic compounds and methods of diagnosing disease or a predisposition to a disease are also provided.

In one aspect, the invention provides the modulation of GPR4 as a therapy for Alzheimer's disease. Modulating GPR4 modulates the expression of genes that are differentially regulated in neurons carrying the E4 genotype compared to homozygous neurons carrying the E3 genotype (E3E3 neurons) including YAP1, VCL, CCND3, CTGF, BDNF and ITGB3. This differential expression is disclosed in FIG. 1.

In certain embodiments of this aspect the subject is homozygous for APOE4 (E4E4 neurons). In other embodiments the subject is heterozygous for the APOE4 allele.

One aspect of the invention provides a method of treatment of a subject diagnosed with, or a predisposition for, Alzheimer's Disease or Mild Cognitive Impairment, said treatment comprising administering a therapeutically effective amount of a molecule that increases the activity of GPR4.

In a preferred embodiment of this aspect, the therapeutically effective amount increases the expression of at least one gene selected from YAP1, CTGF, CCND3, BDNF and VCL or decreases the expression of ITGB3 in a neuronal cell.

In a preferred embodiment of this aspect, a therapy comprises treating a subject with an agonist of GPR4.

In a preferred embodiment of this aspect, a therapy comprises treating a subject with a positive allosteric modulator of GPR4.

In a preferred embodiment of this aspect, the subject is human.

In a preferred embodiment of this aspect, the subject exhibits a decrease in the expression of at least one gene selected from YAP1, CTGF, CCND3, BDNF and VCL or an increase in the expression of ITGB3, compared to a subject who is homozygous for the APOE3 allele.

In a preferred embodiment of this aspect, the therapeutically effective amount increases the expression of at least one gene selected from YAP1, CTGF, CCND3, BDNF and VCL or decreases the expression of ITGB3, to a level comparable to a human who is homozygous for the APOE3 allele.

An aspect of the invention provides a method of measuring the therapeutic effectiveness of a modulator potentially useful in treatment of a subject for Alzheimer's disease or Mild Cognitive Impairment. In a preferred embodiment, the modulation of the differential expression of a gene selected from YAP1, CTGF, CCND3, BDNF, ITGB3 and VCL in a neuronal cell carrying the APOE4 variant versus neuronal cells carrying the APOE3 variant is assessed. The modulator is therapeutically effective if the modulator increases the expression of a gene selected from YAP1, CTGF, CCND3, BDNF and VCL or decreases the expression of ITGB3.

In a preferred embodiment of this aspect, the modulator is a small molecule.

In a preferred embodiment of this aspect, the modulator is an agonist of GPR4.

In a preferred embodiment of this aspect, the modulator is a positive allosteric modulator of GPR4.

In a preferred embodiment of this aspect, the neuronal cell is a human neuronal cell.

In a preferred embodiment of this aspect, the neuronal cell is maintained in a cell culture.

In a preferred embodiment of this aspect, the neuronal cell is located in an organoid.

In a preferred embodiment of this aspect, the neuronal cell is located in a human brain.

In a preferred embodiment of this aspect, the neuronal cell is located in the brain of an individual with Alzheimer's disease or Mild Cognitive Impairment.

In a preferred embodiment of this aspect, the neuronal cell is homozygous for the APOE4 allele.

In a preferred embodiment of this aspect, the subject exhibits a decrease in the expression of at least one gene selected from YAP1, CTGF, CCND3, BDNF and VCL or an increase in the expression of ITGB3, compared to a subject who is homozygous for the APOE3 allele.

In a preferred embodiment of this aspect, the therapeutically effective amount increases the expression of at least one gene selected from YAP1, CTGF, CCND3, BDNF and VCL or decreases the expression of ITGB3, to a level comparable to a human who is homozygous for the APOE3 allele.

An aspect of the invention provides a method of diagnosing a subject heterozygous or homozygous for the APOE4 allele at risk of having Alzheimer's disease or Mild Cognitive Impairment comprising determining the level of expression or activity of an expression product of GPR4 and determining the level of an expression product of at least one gene selected from YAP1, CTGF, CCND3, BDNF, ITGB3 and VCL in a tissue, cell or body fluid of said subject. When for GPR4 the gene expression or activity is decreased, and for at least one of YAP1, CTGF, CCND3, BDNF and VCL a decrease, or for ITGB3 an increase, in the level of the expression product of the gene is seen it is indicative of an elevated risk of Alzheimer's disease or Mild Cognitive Impairment.

In a preferred embodiment of this aspect, the subject is a human.

In a preferred embodiment of this aspect, the subject is homozygous for the APOE4 genotype.

An aspect of the invention provides method for selecting a therapeutic modulator of GPR4 for administration to a subject having, or at-risk of having, Alzheimer's disease or Mild Cognitive Impairment, said method comprising measuring the level of expression of a gene selected from YAP1. CTGF, CCND3, BDNF, ITGB3 and VCL in a biological sample from the subject, and selecting said therapeutic modulator based on whether the subject demonstrates a decrease of the expression product thereof for YAP1, CTGF, CCND3. BDNF or VCL or an increase of the expression product for ITGB3 in said sample.

An aspect of the invention provides a screening assay for identifying a modulator of the expression of a gene or protein selected from among YAP1, CTGF, CCND3, BDNF, VCL and ITGB3 comprising providing cells having an APOE4 allele and measuring the expression of at least one gene or protein selected from YAP1, CTGF, CCND3, BDNF, VCL and ITGB3. The cells are exposed to modulators that are modulators of GPR4 activity and the expression of at least one gene or protein selected from YAP1, CTGF, CCND3, BDNF, VCL and ITGB3 in the cells is measured after the exposure. Modulators that alter the expression of a gene or protein of at least one of YAP1, CTGF, CCND3, BDNF, VCL and ITGB3 are identified.

In a preferred embodiment of this aspect, the modulator is a small molecule.

In a preferred embodiment of this aspect, the cell is a neuronal cell.

In a preferred embodiment of this aspect, the cell is a lymphocyte.

In a preferred embodiment of this aspect, the cell is maintained in a cell culture.

In a preferred embodiment of this aspect, the cell is homozygous for the APOE4 allele.

In a preferred embodiment of this aspect, the neuronal cell is located in an organoid.

In a preferred embodiment of this aspect, the cell is human.

In a preferred embodiment of this aspect, the cell is murine.

In a preferred embodiment of this aspect, the modulator increases the expression of at least one gene selected from YAP1, CTGF, CCND3, BDNF and VCL.

In a preferred embodiment of this aspect, the modulator decreases the expression of ITGB3.

In a preferred embodiment of this aspect, the human neuronal cell is located in the brain of an individual with Alzheimer's disease or Mild Cognitive Impairment

In a preferred embodiment of this aspect, the screen is a high-throughput screen.

### BRIEF DESCRIPTION OF THE DRAWINGS

FIG. 1 shows the relative expression levels of genes YAP1, VCL, CCND3, CTGF, BDNF and ITGB3 in homozygous E4E4 neurons compared to homozygous E3E3 neurons. The relative expression levels are shown as mean±standard error of the mean.

FIG. 2 shows the relative expression levels of genes YAP1, VCL, CCND3, CTGF, BDNF and ITGB3 in homozygous vehicle-treated E4E4 neurons compared to SLC2004-treated homozygous E4E4 neurons and homozygous vehicle-treated E3E3 neurons. The relative expression levels are shown as mean±standard error of the mean.

FIG. 3 shows the relative expression levels of genes YAP1, VCL, CCND3, CTGF, BDNF and ITGB3 in homozygous vehicle-treated E3E3 neurons compared to SLC2002-treated homozygous E3E3 neurons and homozygous vehicle-treated E4E4 neurons. The relative expression levels are shown as mean±standard error of the mean.

### DETAILED DESCRIPTION

GPR4 itself is an APOE4—motif-mediated gene listed in WO2018/112446 that has significantly reduced expression inhuman neurons with the homozygous APOE4 genotype (E4E4 neurons) compared to human neurons with the homozygous APOE3 genotype (E3E3 neurons). The present invention recognizes that modulators of GPR4 (e.g., molecules that increase GPR4 expression level or activity) can be used to restore proper GPR4 activity corresponding to a phenotype observed in normal E3E3 neurons.

GPR4 (G protein coupled receptor 4; UniProtKB as “GPR4_HUMAN” with the accession number P46093). GPR4 is a G protein coupled receptor activated by extracellular acidic pH through the protonation of histidine residues^{xi}. GPR4 signaling progresses through G alpha s (Gₛ)^{xii} allowing activation of the cAMP/EPAC/Rap1 pathway and G alpha 12/13 (G_{12/13})ₓᵢᵢᵢ stimulating the small GTPase RhoA/ROCK. Low pH stimulation of GPR4 play a role in the formation of actin stress fibers^{xiv}, cell adhesion^{xv} and focal adhesion dynamics^{xvi}. Synaptic activity is associated with a transient decrease in the extracellular pH in hippocampal slices^{xvii}. Stimulation of the Gₛ and G_{12′13} pathways enable integrin activation and Yes Associated Protein 1 (YAP1) controlled assembly and maturation of focal adhesions^{xviii} leading to cytoskeletal rearrangements, dendritic spine expansion and synaptic plasticity.

GPR4 influences the expression of genes including YAP1, VCL, CCND3, CTGF, BDNF and ITGB3 resulting in the modulation of gene expression products. A gene expression product comprises a transcription product of such a gene including any RNA transcript based on such gene, including any microRNA or mRNA (whether the mRNA transcript is primary, spliced, edited, modified or mature) or a polypeptide translated from an mRNA transcript. Such polypeptide may be nascent or processed into a mature or modified protein. The amount of expression product may be measured and described qualitatively or quantitatively as an expression level for the product.

YAP1 (Yes Associated Protein 1; UniProtKB as Transcriptional coactivator YAP1, “YAP1_HUMAN” with the accession number P46937) is a downstream effector of the Hippo signaling pathway. YAP1 in complex with transcription factors from the TEA domain family regulate a variety of cellular processes, including cell spreading, proliferation, and migration, glucose uptake and metabolism. YAP1 expression is decreased very early in AD patients before the onset of Aβ deposits or tau tangles^{xix}. YAP1 controls assembly and maturation of focal adhesions and activation of pathways necessary for cytoskeletal rearrangements, dendritic spine expansion and synaptic plasticity.

The link between GPR4 signaling pathways and YAP1 function and expression is uncertain in the literature. In one context, GPR4 mediates the suppression of YAP1^{xx} and in another context, GPR4 signaling promotes YAP1 controlled cell proliferation and survival^{xxi}. The present invention shows that a therapy based on modulating GPR4 leads to the modulation of the expression of YAP1 in the presence of the APOE4 allele.

GPR4 signaling pathways are downregulated by decreased levels of GPR4 as observed in cells carrying the APOE4 allele or by downregulated or inhibited elements of the signaling pathway linking GPR4 to YAP1 function and expression. Genes mutated in carriers of AD familial variants including variants in amyloid precursor protein (APP), presenilin-1 (PSEN1) or presenilin-2 (PSEN2) can be placed in the signaling pathway linking GPR4 to YAP1 function and expression.

GRP4 activity is reduced in cells carrying an APOE4 allele and leads to reduced expression of other genes as described herein. The cascade of reduced expression contributes to a predisposition, or a disease state, for Alzheimer's disease or Mild Cognitive Impairment. It is possible that GPR4 is also reduced in non APOE4 backgrounds when other effectors reducing GPR4 expression are present. In either case, when GRP4 activity is reduced, a therapy based on increasing the activity of GPR4 is useful as a therapy to prevent, delay, reduce or reverse the course of the disease caused by the cascade.

VCL (Vinculin; UniProtKB as “VINC_HUMAN” with the accession number P18206) is a focal adhesion protein that controls focal adhesion formation and integrins dynamics.

CCND3 (Cyclin D3; UniProtKB as G1/S-specific cyclin-D3, “CCND3_HUMAN” with the accession number P30281) is a cell cycle protein and a regulatory subunit of CDK4 and CDK6 kinases. CCND3 accumulates in quiescent cells and is involved in postmitotic arrest^{xxii}.

CTGF/CCN2 (CCN2, Cellular Communication Network Factor 2: UniProtKB as CCN family member 2, “CCN2_HUMAN” with the accession number P29279) is a secreted protein that binds directly to integrins and heparan sulfate proteoglycans hence activating multiple intracellular signaling pathways^{xxiii}.

BDNF (Brain-derived neurotrophic factor; UniProtKB as “BDNF_HUMAN” with the accession number P23560) is a nerve growth factor that promotes neuronal survival. BDNF plays an important function in adult synaptic plasticity. Expression of BDNF is reduced in AD^{xxiv}.

ITGB3 (Integrin Subunit Beta 3; UniProtKB as integrin beta-3, “ITB3_HUMAN” with the accession number P05106) is required for homeostatic synaptic scaling. Adhesion and surface level of P3 integrin in postsynaptic neurons directly correlates with synaptic strength and the abundance of synaptic GLUR2 (GRIA2, Glutamate Ionotropic Receptor AMPA Type Subunit 2; UniProtKB as Glutamate receptor 2, “GRIA2_HUMAN” with the accession number P42262), an AMPAR subunit^{xxv}.

Modulators of GPR4 include molecules that alter the activity of GPR4. Modulators include molecules that are agonists or antagonists of GPR4. A GPR4 agonist increases the activity of GPR4 and a GPR4 antagonist decreases the activity of GPR4. Molecules include small molecule chemical compounds and large molecule biological compounds. Biological compounds include RNA, DNA, antibodies and antigen binding fragments of antibodies containing complementarity determining regions (CDRs) specific for GPR4 or a protein or a co-factor that interact with GPR4.

A small molecule is an organic compound with a molecular weight of less than 2000 Dalton, preferably less than 1500 Dalton and most preferably 900 Dalton or less.

A GPR4 agonist increases the activity of GPR4 and thereby influences the expression level of at least one gene selected from YAP1, VCL, CCND3, CTGF, BDNF and ITGB3; For YAP1, VCL, CCND3, CTGF, BDNF the GPR4 agonist effect is an increase in the expression level and for ITGB3 the GPR4 agonist effect is a decrease in the expression level.

Exemplary antagonists of GPR4 are known to those skilled in the art. SLC2002 is an exemplary antagonist of GPR4 and referred to as compound 3b in the cited reference^{xxvi}. SLC2002 is a potent and selective GPR4 antagonist.

As used herein, a “GPR4 agonist” is a compound that binds to GPR4 (or to a protein or co-factor that interacts directly with GPR4) and/or causes an increase of the GPR4 cellular activity. The activity of a GPR4 agonist can include the activation of the G. (cAMP) and/or G_{12/13} signaling pathways, a shift of the pH activation curve of the GPR4 receptor required to induce the protonation of histidine residues, the control of the oligomerization of GPR4 or the association of GPR4 with interaction partners, or affect the endocytosis or desensitization of GPR4. These GPR4 activation mechanisms serve as examples and other events directly linked to GPR4 activation are known to the one skilled in the art. SLC2004, sphingosylphosphorylcholine, is an exemplary agonist of GPR4^{xxvii}. Another known agonist of GPR4 is lysophosphatidylcholine^{xxviii}.

Positive allosteric modulators of GPR4 are agonists of GPR4 that increase the activity of GPR4 through binding to a site on GPR4 that is different from the GPR4 ligand binding site.

The invention includes diagnostic assays for the determination whether a subject has, or is at risk of, Alzheimer's disease or Mild Cognitive Impairment. Subjects are mammals and preferable are human subjects. According to the present invention, an assay is performed to determine if the subject carries an APOE4 allele. If so, then one or more assays is performed to determine the level of expression or activity of GPR4 and the expression of at least one gene selected from YAP1, VCL, CCND3, CTGF, BDNF and ITGB3. If the subject exhibits a decrease in GPR4 expression or activity and a decrease in the expression of at least one of YAP1, VCL, CCND3, CTGF, BDNF, or an increase in ITGB3 expression, then the subject has, or is at risk, of Alzheimer's disease or Mild Cognitive Impairment. In a preferred embodiment, the expression of YAP1 is measured in addition to the measure(s) of GPR4.

Tissues, cells or body fluids from subjects are collected for diagnostic assays. Measures of protein or mRNA expression levels in tissues, cells or body fluids are obtained and compared. In a preferred embodiment, the tissue collected from subjects for analysis includes whole blood. In another preferred embodiment, fluids collected from subjects for analysis include blood plasma, blood serum, sputum, saliva, sweat, urine, lymph or cerebrospinal fluid. In yet another preferred embodiment, cells collected from subjects include blood cells, buccal cells, skin fibroblasts, neuronal cells or lymphocytes. In a most preferred embodiment, the cells are neuronal cells.

The present invention includes screening assays for the identification of therapeutic agents useful in the treatment of Alzheimer's disease and Mild Cognitive Impairment. Screening assays are conducted to measure the effects of molecules on the expression or activity of GPR4 and expression of at least one gene selected from YAP1, VCL, CCND3, CTGF, BDNF and ITGB3. Screening assays can use cells, particularly neuronal cells, or can be cell free. The cells can be derived from human subjects or animal subjects.

Screening assays of the invention are designed to identify modulation effects of molecules on the expression or activity levels of GPR4 and at least one gene selected from YAP1, VCL, CCND3, CTGF, BDNF and ITGB3. As used herein the term “modulation” means any change in activity of a function or amount of the transcribed gene, mRNA or protein, including any change in transcription rate or expression level. The term “change” when referring to a level of a biological activity or expression level, means the value is statistically different from a control (i.e., p<0.25, often p<0.1, and more often p<0.05). The term “control” of gene expression or activity of a gene expression product refers to a standard level against which gene expression or the activity of the gene expression product, respectively, in a cell is or can be compared.

Preferred assays are optimized for speed, efficiency, signal detection and low reagent consumption. (Zhang et al. (1999) J. Biomolec. Screen. 4(2):67). Assays can be developed using a variety of cells or cell extracts, preferably but not limited to neuronal cells, neuronal progenitor cells, differentiated neurons, oligodendrocytes, fibroblasts, lymphocytes, human embryonic kidney cells or another cell type or extracts thereof.

In some embodiments, the screening assays of the invention, either cellular, cell extract or biochemically based with substantially purified genes or gene expression products, are designed for testing a plurality of compounds (e.g., millions) through high-throughput screening of chemical libraries.

Chemical libraries of test compounds that may be screened to identify a modulator can be obtained from numerous available resources or using any of the numerous approaches in library synthesis methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). See also Dolle et al. (2010) Comprehensive Survey of Chemical Libraries for Drug Discovery and Chemical Biology: 2009. J. Comb. Chem., 2010, 12 (6), pp 765-806.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Test compounds which successfully modulate the activity and/or expression level of GPR4 and the level of expression of at least one gene selected from YAP1, VCL, CCND3, CTGF, BDNF and ITGB3 are attractive candidates for further investigation and secondary screening in alternative assays for potential use in treating AD or Mild Cognitive Impairment. Compounds are considered “potentially useful for treatment” when first identified in a screening assay, because it is well known that initial successful hits rarely contain all the required features for a successful pharmaceutical. They are however extremely useful to allow researchers to identify a chemical core structure shared among compounds that effectively modulates the target activity. Typically, when a core structure is identified, an extensive library of possibly thousands of related compounds is further developed with the aim of identifying a lead compound that meets all the criteria for a successful pharmaceutical candidate. The assay is used repeatedly through many rounds of screening of up to millions of compounds to ultimately identify a small group of lead compounds, one of which may eventually become an approved therapeutic agent.

In some embodiments of the invention, a possible lead molecule for treatment of AD or Mild Cognitive Impairment identified by methods of the invention exhibits a 50% activation concentration (EC₅₀) of about 500 μM or less, typically about 100 μM or less, often about 50 μM or less, more often about 10 μM or less, and most often about 500 nM or less.

The present invention provides methods of treatment of subjects with Alzheimer's disease or Mild Cognitive Impairment. Clinical use of methods of the invention includes a method for treating a subject suffering from Alzheimer's disease or Mild Cognitive Impairment.

The molecule of the invention can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the molecule of the invention may be incorporated with excipient and used in the form of ingestible tablets, buccal tablets, troches, capsules, elixirs, suspensions, syrups, wafers, and the like. Such compositions and preparation can contain at least 0.1% of molecule of the invention. The percentage of the compositions and preparations can be varied and can conveniently be between about 1 to about 10% of the weight of the unit. The amount of molecule of the invention in such therapeutically useful compositions is such that a suitable dosage will be obtained. Preferred compositions or preparations according to the present invention are prepared such that an oral dosage unit form contains from about 1 to about 1000 mg of the molecule of the invention.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the molecule of the invention, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the molecule of the invention can be incorporated into sustained-release preparations and formulation.

The molecule of the invention can also be administered parenterally. Solutions of the molecule of the invention as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the molecule of the invention in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The physician will determine the dosage of the molecule of the invention which will be most suitable for prophylaxis or treatment and it will vary with the form of administration and the particular molecule chosen, and also, it will vary with the particular subject under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

The present invention provides for methods for selecting a therapeutic modulator of GPR4 for administration to a subject having, or at-risk of having, Alzheimer's disease or Mild Cognitive Impairment. The method comprises of measuring the level of expression of a gene selected from YAP1, CTGF, CCND3, BDNF, ITGB3 and VCL in a biological sample from the subject and selecting said therapeutic modulator of GPR4 based on whether the subject demonstrates a decrease of the expression product thereof for YAP1, CTGF, CCND3, BDNF or VCL or an increase of the expression product for ITGB3 in said sample. The biological sample includes tissues, cells or body fluids from said subject. In a preferred embodiment, the tissue collected from subjects for analysis includes whole blood. In another preferred embodiment, fluids collected from subjects for analysis include blood plasma, blood serum, sputum, saliva, sweat, urine, lymph or cerebrospinal fluid. In yet another preferred embodiment, cells collected from subjects include blood cells, buccal cells, skin fibroblasts, neuronal cells or lymphocytes.

### EXAMPLES

Example 1: Cultivation of human isogenic neurons carrying either the homozygous APOE4 genotype (E4E4) or the homozygous APOE3 genotype (E3E3). Commercially available human isogenic neurons were acquired from Fujifilm Cellular Dynamics Inc. The homozygous APOE4 genotype (E4E4) neurons were designated DDP-NRC-1× 01434.779 and herein they are referred to as E4E4 neurons. The homozygous APOE3 genotype (E3E3) neurons were designated R1013 and herein they are referred to as E3E3 neurons. The neurons were plated and cultivated in 96-well plates. Prior to plating of neurons, the manufacturer supplied complete maintenance medium was prepared and stored at 4 degrees Celsius. The 96-well plates were double coated with poly-L-ornithin (PLO Sigma; P4957) and laminin (Sigma; L2020) by first coating the wells with poly-L-ornithin overnight at 4 degrees Celsius. Wells were rinsed with phosphate-buffered saline and then laminin diluted in phosphate-buffered saline at a concentration of 10 micrograms/milliliter was added for three hours. Complete maintenance medium was equilibrated to room temperature. Neurons were thawed for three minutes in a 37 degrees Celsius water bath. The content of the vial was transferred to a 50 ml conical tube by adding one milliliter of room-temperature equilibrated complete maintenance medium and suspending the neurons. The neuron suspension was transferred to the 50 ml conical tube. This neuron suspension was further diluted by addition of eight milliliter of complete maintenance medium. A viable neuron count was performed and the neurons were plated into individual wells of a 96-well plate at a neuronal seed density of 125,000 neurons per cm². The E3E3 and the E4E4 containing 96-well plates were transferred into a cell incubator and incubated at 37 degrees Celsius and 5% CO₂. E3E3 and E4E4 neurons plated in this way were allowed to attach to the wells of the 96-well plate for 24 hours at which time a 100% medium change to fresh complete maintenance medium was performed. The E3E3 and E4E4 neurons were then continued to be cultivated at 37 degrees Celsius and 5% CO₂. On Day 5 after plating a 50% medium change was performed by removal of 50% of the cell supernatant and addition of the same volume of fresh complete maintenance medium and then the 96-well plates were continued to be cultivated at 37 degrees Celsius and 5% CO₂. On Day 9 after plating, a 50% medium change was performed by removal of 50% of the cell supernatant and addition of the same volume of fresh complete maintenance medium. This medium change was performed before neurons were treated with compounds on Day 9 after plating.

Example 2: Treatment of E4E4 neurons with the exemplary GPR4 agonist SLC2004 and vehicle and E3E3 neurons with vehicle. E4E4 and E3E3 neurons were plated and cultivated in individual wells of a 96-well plate as described in Example 1. The compound spingosylphosphorylcholine (SLC2004) (Sigma; S4257) is a lyophilized powder with a white to yellow appearance and a molecular weight of 464.62 Dalton. A fresh stock solution of SLC2004 was prepared in methanol as vehicle at a stock concentration of 300 millimolar. This stock solution was further diluted to yield two 21-fold concentrated solutions of the intended in-well concentrations. For one of the 21-fold concentrated SLC2004 solution, a final SLC2004 in-well concentration of 3 micromolar was achieved by adding 10 microliters of this 21-fold concentrated SLC2004 solution to 200 microliters of complete maintenance medium in each well of the 96-well plate. For the second 21-fold concentrated SLC2004 solution, a final SLC2004 in-well concentration of 10 micromolar was achieved by adding 10 microliters of this 21-fold concentrated SLC2004 solution to 200 microliters of complete maintenance medium in each well of the 96-well plate. All wells with SLC2004-treated neurons contained the same amount of 0.01% methanol in complete maintenance medium. In parallel, E4E4 neurons and E3E3 neurons were cultivated in 0.01% methanol in complete maintenance medium in the absence of SLC2004. These samples served as vehicle controls. E4E4 neurons treated with SLC2004 or vehicle (0.01% methanol in complete maintenance medium) and E3E3 neurons treated with vehicle (0.01% methanol in complete maintenance medium) were incubated for six hours at 37 degrees Celsius at 5% CO₂.

Example 3: Treatment of E3E3 neurons with the exemplary GPR4 antagonist SLC2002 and vehicle and E4E4 neurons with vehicle. E4E4 and E3E3 neurons were plated and cultivated in individual wells of a 96-well plate as described in Example 1. The compound SLC2002 is a lyophilized powder with a white to yellow appearance and a molecular weight of 479.66 Dalton. A fresh stock solution of SLC2002 was prepared in methanol at a stock concentration of 100 millimolar. This stock solution was further diluted to yield a 21-fold concentrated solution. This SLC2002 solution was used to end up with a final SLC2002 in-well concentration of 1 micromolar by adding 10 microliters of this 21-fold concentrated SLC2002 solution to 200 microliters of complete maintenance medium in each well of the 96-well plate. All wells with SLC2002-treated neurons contained the same amount of vehicle (0.01% methanol in complete maintenance medium). In parallel, E4E4 neurons and E3E3 neurons were cultivated with vehicle (0.01% methanol in complete maintenance medium) in the absence of SLC2002. These samples served as vehicle controls. E3E3 neurons treated with SLC2002 or vehicle (0.01% methanol in complete maintenance medium) and E4E4 neurons treated with vehicle (0.01% methanol in complete maintenance medium) were incubated for twelve hours at 37 degrees Celsius at 5% CO₂.

Example 4: Collection of treated E4E4 and E3E3 neurons for analysis of GPR4 agonist activity. Neuronal material for the later analysis of SLC2004 effects on E4E4 neurons was generated by incubating E4E4 neurons for six hours with SLC2004 or vehicle and E3E3 neurons for six hours with vehicle as described in Example 2. At the end of the six hours incubation period, the complete maintenance medium was removed from each well of the 96-well plate. The neurons were detached from each well in 100 microliters of phosphate-buffered saline, transferred to a fresh tube and centrifuged for three minutes at 225 g. The phosphate-buffered saline was aspirated down to about 15 microliters and the neuron pellets were snap frozen on dry ice and stored at −80 degrees Celsius until analysis.

Example 5: Collection of treated E4E4 and E3E3 neurons for analysis of GPR4 antagonist activity. Neuronal material for the later analysis of SLC2002 effects on E3E3 neurons was generated by incubating E3E3 neurons for twelve hours with SLC2002 or vehicle and E4E4 neurons for twelve hours with vehicle as described in Example 3. At the end of the twelve hours incubation period, the complete maintenance medium was removed from each well of the 96-well plate. The neurons were detached from each well in 100 microliters of phosphate-buffered saline, transferred to a fresh tube and centrifuged for three minutes at 225 g. The phosphate-buffered saline was aspirated down to about 15 microliters and the neuron pellets were snap frozen on dry ice and stored at −80 degrees Celsius until analysis.

Example 6: Quantitative polymerase chain reaction assays to determine expression levels of genes responding to GPR4 based therapy. Total RNA was extracted from each of the snap frozen neuron pellets described in Examples 4 and 5 using the Direct-zol™ RNA MiniPrep Kit (Zymo Research, Irvine, CA; Cat. no. R2050) according to the manufacturer's instructions with optional on-column DNase treatment. Total RNA was extracted from E4E4 neurons treated with the exemplary agonist SLC2004 or vehicle as well as E3E3 neurons treated with vehicle after a six-hour incubation. Total RNA was also extracted from E3E3 neurons treated with the exemplary antagonist SLC2002 or vehicle as well as E4E4 neurons treated with vehicle after a twelve-hour incubation. RNA concentration and purity were determined using a Nanodrop ND-1000 spectrophotometer (Thermo Scientific). RNA integrity was measured using the Agilent 2100 Bioanalyzer (Agilent Technologies, Palo Alto, CA) and the RNA 6000 Nano Chip Kit according to the manufacturer's instructions. Subsequently, 3 micrograms of total RNA were used as template to synthesize cDNA with the High Capacity cDNA Reverse Transcription Kit (Applied Biosystems, Foster City, CA; Cat. no. 4368814). Detection of polymerase chain reaction (PCR) products is enabled by the use of a fluorescent reporter molecule in the reaction that yields increased fluorescence with an increasing amount of product DNA. A method of detection was employed that involves the double-stranded DNA intercalating molecule SYBR Green® to determine gene expression levels of protein encoding genes YAP1, BDNF, CTGF, CCND3, VCL and ITGB3. Real time PCR was performed on the BioRad CFX384 Real Time System (BioRad, Hercules, CA) using forward and reverse amplification primers specifically designed to detect each of the genes of interest YAP1, BDNF, CTGF, CCND3, VCL and ITGB3. Each reaction well contained 5 microliters of PowerUp™ SYBR Green Master Mix (Applied Biosystems; Cat. no. A25742), cDNA equivalent to 13 ng of total RNA and 250 nM each of forward and reverse amplification primers in a final reaction volume of 10 microliters. In this invention, primers for all assays were designed using Primer 3^{xxix} and QuantPrime^{xxx}. A melting curve analysis was performed to ensure single-product amplification for all primer pairs. Cycling conditions were as follows: 95° C. for 10 minutes for polymerase activation, followed by 40 cycles of 95° C. for 15 seconds and 60° C. for 1 minute. Cytochrome c1 (CYC1) and ribosomal protein L13 (RPL13) were used as reference genes. Each quantitative qPCR experiment including amplification and data reduction was performed in triplicates. Data analysis was performed using CFX Manager software from BioRad, version 3.1. The experimental Cq (cycle quantification) was calibrated against the endogenous control products. Quantification of nucleic acids was achieved using relative quantification analysis (Double delta Ct data analysis, ΔΔCt method). Relative quantification allows to determine fold-differences in expression of the target gene in the test cell line relative to the control cell line. First, a baseline was subtracted from the raw data based on the raw fluorescence values. Next, the threshold cycle (Ct) value was determined for each sample, which represents the number of cycles needed to reach a particular quantification threshold fluorescence signal level. Ct values were determined both for the genes being evaluated and for reference genes for normalization purposes. Average Ct values were determined for a gene of interest and the reference genes (designated as “Ref”) in all neuronal samples.

Example 7: Determination of exemplary GPR4 agonist effects on the expression genes in E4E4 neurons. For paired comparisons the following four values were generated for SLC2004 treated E4E4 samples and the E4E4 vehicle controls: Avg. Ct Ref in E4E4 treated with SLC2004, Avg. Ct Ref in E4E4 treated with vehicle, Avg. Ct gene of interest in E4E4 treated with SLC2004, and Avg. Ct gene of interest in E4E4 treated with vehicle. The differences between Ct values of the gene of interest and reference genes (delta Ct values, short dCt) were calculated for the E4E4 treated with SLC2004 and the E4E4 treated with vehicle. Next, the difference between E4E4 treated with SLC2004 and E4E4 treated with vehicle was calculated to arrive at the Double Delta Ct Value (ddCt E4E4 treated with SLC2004—E4E4 treated with vehicle). Since the quantity of amplified product doubles in each cycle, the expression fold change between E4E4 treated with SLC2004 and E4E4 treated with vehicle (Relative Quantification or RQ (E4E4 treated with SLC2004/E4E4 treated with vehicle)) was computed with the following formula 2\hat{ }^{ddCt}. E3E3 neurons treated with vehicle were processed in the same way as E4E4 treated with SLC2004 to yield a relative expression level of genes of interest compared to vehicle-treated E4E4 neurons. Statistical analyses were performed by using 1-way ANOVA followed by Dunnett's test post-hoc to account for multiple comparisons.

Example 8: Determination of exemplary GPR4 antagonist effects on the expression of genes in E3E3 neurons. For paired comparisons the following four values were generated for SLC2002 treated E3E3 samples and the E3E3 vehicle controls: Avg. Ct Ref in E3E3 treated with SLC2002, Avg. Ct Ref in E3E3 treated with vehicle. Avg. Ct gene of interest in E3E3 treated with SLC2002, and Avg. Ct gene of interest in E3E3 treated with vehicle. The differences between Ct values of the gene of interest and reference genes (delta Ct values, short dCt) were calculated for the E3E3 treated with SLC2002 and the E3E3 treated with vehicle. Next, the difference between E3E3 treated with SLC2002 and E3E3 treated with vehicle was calculated to arrive at the Double Delta Ct Value (ddCt E3E3 treated with SLC2002—E3E3 treated with vehicle). Since the quantity of amplified product doubles in each cycle, the expression fold change between E3E3 treated with SLC2002 and E3E3 treated with vehicle (Relative Quantification or RQ (E3E3 treated with SLC2002/E3E3 treated with vehicle)) was computed with the following formula 2\hat{ }^{ddCt}. E4E4 neurons treated with vehicle were processed in the same way as E3E3 treated with SLC2002 to yield a relative expression level of genes of interest compared to vehicle-treated E3E3. Statistical analyses were performed by using 1-way ANOVA followed by Dunnett's test post-hoc to account for multiple comparisons.

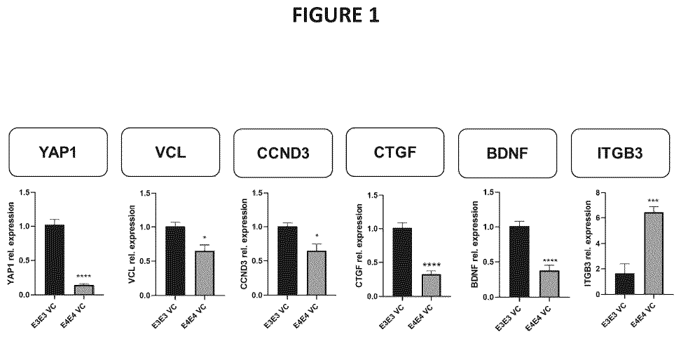

Example 9: Gene expression in human isogenic neurons carrying the E4E4 and the E3E3 genotypes. Samples generated as disclosed in previous examples were analyzed for expression levels of genes as disclosed in Examples 6, 7 and 8 These genes included YAP1, VCL, CCND3, CTGF, BDNF and ITGB3. The differential expression of these genes in E4E4 neurons compared to E3E3 neurons is shown in FIG. 1. The relative expression levels are shown as mean±standard error of the mean. Statistical analysis was performed by unpaired two-tailed t tests comparing expression levels of these genes in E3E3 neurons and E4E4 neurons. P-values are shown as asterisks with the following meanings: (*) p≤5.05, (**) p≤0.01, (***) p≤0.0001 and (****) p≤0.0001. The comparison of E4E4 vehicle-treated (E4E4 VC) and E3E3 vehicle-treated (E3E3 VC) neurons revealed that the expression of YAP1, VCL, CCND3. CTGF and BDNF was downregulated in E4E4 vehicle-treated (E4E4 VC) neurons compared to E3E3 vehicle-treated (E3E3 VC) neurons. The expression of ITGB3 was upregulated in E4E4 vehicle-treated (E4E4 VC) compared to E3E3 vehicle-treated (E3E3 VC) neurons.

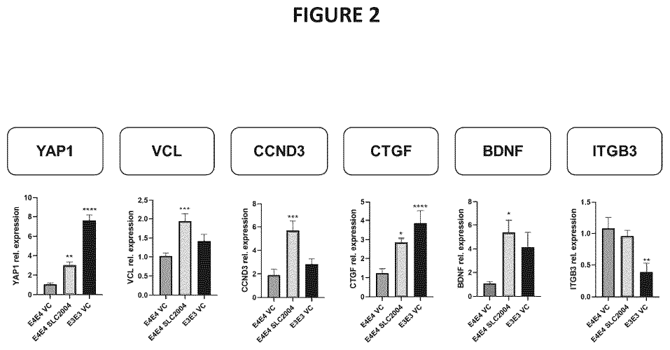

Example 10: Treatment of E4E4 neurons with the exemplary GPR4 agonist SLC2004 modulates the expression of genes. Samples generated as disclosed in previous examples were analyzed for expression levels of genes as disclosed in Examples 7. These genes included YAP1, VCL, CCND3, CTGF, BDNF and ITGB3. The differential expression of these genes in E4E4 vehicle-treated neurons compared to E4E4 SLC2004-treated neurons is shown in FIG. 2. The relative expression levels are shown as mean±standard error of the mean. E3E3 vehicle-treated neurons are shown for comparison. Statistical analysis was performed by 1-way ANOVA followed by Dunnett's multiple comparisons test comparing expression levels of these genes in E4E4 vehicle-treated neurons compared to E4E4 SLC2004-treated neurons and E3E3 vehicle-treated neurons. P-values are shown as asterisks with the following meanings: (*) p≤0.05, (**) p≤0.01, (***) p≤0.001 and (****) p≤0.0001. The comparison of E4E4 neurons treated with SLC2004 and E4E4 vehicle-treated neurons revealed that the expression of YAP1. CTGF and BDNF was upregulated after 10 micromolar SLC2004 treatment (FIG. 2). The comparison of E4E4 neurons treated with SLC2004 and E4E4 vehicle-treated neurons revealed that the expression of VCL and CCND3 was upregulated after 3 micromolar SLC2004 treatment (FIG. 2). ITGB3 was downregulated after 3 micromolar SLC2004 treatment (FIG. 2).

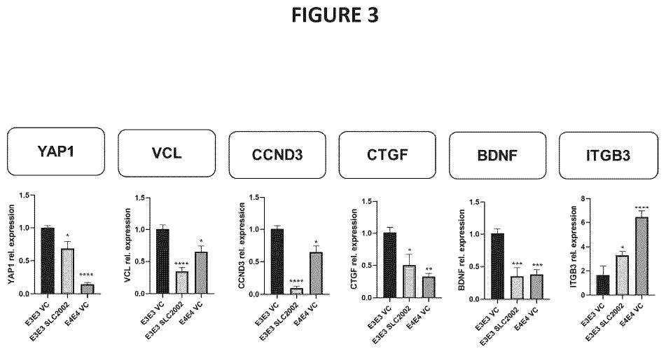

Example 11: Treatment of E3E3 neurons with the exemplary GPR4 antagonist SLC2002 modulates the expression of genes. Samples generated as disclosed in previous examples were analyzed for expression levels of genes as disclosed in Example 8. These genes included YAP1, VCL, CCND3, CTGF, BDNF and ITGB3. The differential expression of these genes in E3E3 vehicle-treated neurons compared to E3E3 SLC2002-treated neurons is shown in FIG. 3. The relative expression levels are shown as mean±standard error of the mean. E4E4 vehicle-treated neurons are shown for comparison. Statistical analysis was performed by 1-way ANOVA followed by Dunnett's multiple comparisons test comparing expression levels of genes YAP1, VCL, CCND3, CTGF and BDNF in E3E3 vehicle-treated neurons compared to E3E3 SLC2002-treated neurons. The statistical analysis of ITGB3 levels in E3E3 vehicle treated neurons compared to E3E3 SLC2002-treated neurons and E4E4 vehicle-treated neurons was performed by 1-way ANOVA followed by Fisher's Least Significant Difference test. P-values are shown as asterisks with the following meanings: (*) p≤0.05, (**) p≤0.01, (***) p≤0.001 and (****) p≤0.0001. The comparison of E3E3 neurons treated with SLC2002 and E3E3 vehicle-treated neurons revealed that the expression of YAP1, VCL, CCND3, CTGF and BDNF was downregulated after 1 micromolar SLC2002 treatment (FIG. 3). ITGB3 was upregulated after 1 micromolar SLC2002 treatment (FIG. 3).

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### REFERENCES CITED

ⁱBenarroch E E, Glutamatergic synaptic plasticity and dysfunction in Alzheimer disease: Emerging mechanisms. Neurology. 2018 Jul. 17; 91(3):125-132.
ⁱⁱCarter T L, Rissman R A, Mishizen-Eberz A J, Wolfe B B, Hamilton R L, Gandy S, Armstrong D M. Differential preservation of AMPA receptor subunits in the hippocampi of Alzheimer's disease patients according to Braak stage. Exp Neurol. 2004 June; 187(2):299-309.
ⁱⁱⁱZekanowski C, Wojda U. Aneuploidy, chromosomal missegregation, and cell cycle reentry in Alzheimer's disease. Acta Neurobiol Exp (Wars). 2009; 69(2):232-53.
^{iv}Kittur S, Hoh J, Endo H, Tourtellotte W, Weeks B S, Markesbery W, Adler W. Cytoskeletal neurofilament gene expression in brain tissue from Alzheimer's disease patients. I. Decrease in NF-L and NF-M message. J Geriatr Psychiatry Neurol. 1994 July-September; 7(3):153-8.
^{v}Ma, Q. L., Galasko, D. R., Ringman, J. M., Vinters, H. V., Edland, S. D., Pomakian, J., Ubeda, O. J., Rosario, E. R., Teter, B., Frautschy, S. A. et al. (2009). Reduction of SorLA/LR11, a sorting protein limiting beta-amyloid production, in Alzheimer disease cerebrospinal fluid. Arch. Neurol. 66.448-457.
^{vi}Raber J. Huang Y, Ashford J W. ApoE genotype accounts for the vast majority of AD risk and AD pathology. Neurobiol Aging. 2004 May-June; 25(5):641-50.
^{vii}Farrer L A, Cupples L A, Haines J L, Hyman B, Kukull W A, Mayeux R, Myers R H, Pericak-Vance M A, Risch N, van Duijn C M. Effects of age, sex, and ethnicity on the association between apolipoprotein E genotype and Alzheimer disease. A meta-analysis. APOE and Alzheimer Disease Meta Analysis Consortium. JAMA. 1997 Oct. 22-29; 278(16):1349-56.
^{viii}Urfer-Buchwalder A, Urfer R. Identification of a Nuclear Respiratory Factor 1 Recognition Motif in the Apolipoprotein E Variant APOE4 linked to Alzheimer's Disease. Sci Rep. 2017 Jan. 17; 7:40668.
^{ix}Johar K, Priya A, Wong-Riley M T. Regulation of Na(+)/K(+)-ATPase by nuclear respiratory factor 1: implication in the tight coupling of neuronal activity, energy generation, and energy consumption. J Biol Chem. 2012 Nov. 23; 287(48):40381-90. doi: 10.1074/jbc.M112.414573. Epub 2012 Oct. 9.
^{x}WO 2018/112446 A2: Use of APOE4 motif-mediated genes for diagnosis and treatment of Alzheimer's disease.
^{xi}Tobo M, Tomura H, Mogi C, Wang J Q, Liu J P, Komachi M, Damirin A, Kimura T, Murata N, Kurose H, Sato K, Okajima F. Previously postulated “ligand-independent” signaling of GPR4 is mediated through proton-sensing mechanisms. Cell Signal. 2007 August; 19(8) 1745-53. Epub 2007 Mar. 30.
^{xii}Ludwig M G, Vanek M, Guerini D, Gasser J A, Jones C E, Junker U, Hofstetter H, Wolf R M, Seuwen K. Proton-sensing G-protein-coupled receptors. Nature. 2003 Sep. 4; 425(6953):93-8.
^{xiii}Justus C R, Yang L V. GPR4 decreases B16F10 melanoma cell spreading and regulates focal adhesion dynamics through the G13/Rho signaling pathway. Exp Cell Res. 2015 May 15; 334(1):100-13.
^{xiv}Castellone R D, Leffler N R, Dong L, Yang L V. Inhibition of tumor cell migration and metastasis by the proton-sensing GPR4 receptor. Cancer Lett. 2011 Dec. 22; 312(2):197-208.
^{xv}Chen A, Dong L, Leffler N R, Asch A S, Witte O N, Yang L V. Activation of GPR4 by acidosis increases endothelial cell adhesion through the cAMP/Epac pathway. PLoS One. 2011; 6(11):e27586.
^{xvi}Justus C R, Yang L V. GPR4 decreases B16F10 melanoma cell spreading and regulates focal adhesion dynamics through the G13/Rho signaling pathway. Exp Cell Res. 2015 May 15; 334(1):100-13.
^{xvii}Krishtal O A, Osipchuk Y V, Shelest T N, Smirnoff S V. Rapid extracellular pH transients related to synaptic transmission in rat hippocampal slices. Brain Res. 1987 dec 15; 436(2):352-6.
^{xviii}Nardone G, Oliver-De La Cruz J, Vrbsky J, Martini C, Pribyl J, Skládal P, Pex̆l M, Caluori G, Pagliari S, Martino F, Maceckova Z, Hajduch M, Sanz-Garcia A, Pugno N M, Stokin G B, Forte G. YAP regulates cell mechanics by controlling focal adhesion assembly. Nat Commun. 2017 May 15; 8:15321.
^{xiv}Xu M, Zhang D F, Luo R, Wu Y, Zhou H, Kong L L, Bi R, Yao Y G. A systematic integrated analysis of brain expression profiles reveals YAP1 and other prioritized hub genes as important upstream regulators in Alzheimer's disease. Alzheimers Dement. 2018 Fcb; 14(2):215-229.
^{xv}Tao S C, Gao Y S, Zhu H Y, Yin J H, Chen Y X, Zhang Y L, Guo S C, Zhang C Q. Decreased extracellular pH inhibits osteogenesis through proton-sensing GPR4-mediated suppression of yes-associated protein. Sci Rep. 2016 Jun. 3; 6:26835.
^{xvi}Yu M, Cui R, Huang Y, Luo Y, Qin S, Zhong M. Increased proton-sensing receptor GPR4 signalling promotes colorectal cancer progression by activating the hippo pathway. EBioMedicine. 2019 October; 48:264-276.
^{xvii}Lacomme M, Liaubet L, Pituello F, Bel-Vialar S. NEUROG2 drives cell cycle exit of neuronal precursors by specifically repressing a subset of cyclins acting at the G1 and S phases of the cell cycle. Mol Cell Biol. 2012 July; 32(13):2596-607.
^{xviii}Malik A R. Liszewska E, Jaworski J. Matricellular proteins of the Cyr61/CTGF/NOV (CCN) family and the nervous system. Front Cell Neurosci. 2015 Jun. 24; 9:237.
^{xxiv}Lu B, Nagappan G, Lu Y. BDNF and synaptic plasticity, cognitive function, and dysfunction. Handb Exp Pharmacol. 2014; 220:223-50.
^{xvx}Pozo K, Cingolani L A, Bassani S, Laurent F, Passafaro M, Goda Y. β3 integrin interacts directly with GluA2 AMPA receptor subunit and regulates AMPA receptor expression in hippocampal neurons. Proc Natl Acad Sci USA. 2012 Jan. 24; 109(4):1323-8.
^{xxvi}Fukuda H, Ito S, Watari K, Mogi C, Arisawa M, Okajima F, Kurose H, Shuto S. Identification of a Potent and Selective GPR4 Antagonist as a Drug Lead for the Treatment of Myocardial Infarction. ACS Med Chem Lett. 2016 Feb. 24; 7 (5):493-7.
^{xxvii}Kim K S, Ren J, Jiang Y, Ebrahem Q, Tipps R, Cristina K, Xiao Y J, Qiao J, Taylor K L, Lum H, Anand-Apte B, Xu Y. GPR4 plays a critical role in endothelial cell function and mediates the effects of sphingosylphosphorylcholine. FASEB J. 2005 May; 19(7):819-21. Epub 2005 Feb. 17.
^{xxviii}Qiao J, Huang F, Naikawadi R P, Kim K S, Said T, Lum H. Lysophosphatidylcholine impairs endothelial barrier function through the G protein-coupled receptor GPR4. Am J Physiol Lung Cell Mol Physiol. 2006 July; 291(1):L91-101. Epub 2006 Feb. 3.
^{xxix}Ye, J., Coulouris, G., Zaretskaya, I., Cutcutache, I., Rozen, S., & Madden, T. L. (2012). Primer-BLAST: a tool to design target-specific primers for polymerase chain reaction. BMC bioinformatics, 13(1), 134.
^{xxx}Arvidsson, S., Kwasmewski, M., Riaño-Pachón, D. M., & Mueller-Roeber, B. (2008). QuantPrime—a flexible tool for reliable high-throughput primer design for quantitative PCR. BMC bioinformatics, 9(1), 465.

What is claimed is:

1. A method of treatment of a subject diagnosed with, or a predisposition to, Alzheimer's Disease or Mild Cognitive Impairment, said treatment comprising:
administering a therapeutically effective amount of a molecule that increases the activity of GPR4,
The method of claim 1 wherein said administration increases the expression of at least one gene selected from YAP1, CTGF, CCND3, BDNF and VCL or decreases the expression of ITGB3 in the subject.

2. The method of claim 1 in which the molecule is an agonist of GPR4.

3. The method of claim 1 in which the molecule is a positive allosteric modulator of GPR4.

4. The method of claim 1 in which the subject is human.

5. The method of claim 1 in which the subject carries the APOE4 allele.

6. The method of claim 5 wherein the subject exhibits a decrease in the expression of at least one gene selected from YAP1, CTGF, CCND3, BDNF and VCL or an increase in the expression of ITGB3, compared to a human who is homozygous for the APOE3 allele.

7. The method of claim 6 wherein the therapeutically effective amount increases the expression of at least one gene selected from YAP1, CTGF, CCND3, BDNF and VCL or decreases the expression of ITGB3, to a level comparable to a human who is homozygous for the APOE3 allele.

8. A method of measuring the therapeutic effectiveness of a modulator of GPR4 potentially useful in treatment of a subject for Alzheimer's disease or Mild Cognitive Impairment by determining the effect of the modulator on the differential expression of a gene selected from YAP1, CTGF, CCND3, BDNF, ITGB3 and VCL in a neuronal cell carrying the APOE4 allele versus a neuronal cell homozygous for the APOE3 allele, wherein the modulator is therapeutically effective if the modulator increases the expression of a gene selected from YAP1, CTGF, CCND3, BDNF and VCL or decreases the expression of ITGB3.

9. The method of claim 8 in which the neuronal cell is homozygous for the APOE4 allele.

10. The method of claim 8 wherein the modulator is a small molecule.

11. The method of claim 8 wherein the modulator is an agonist of GPR4.

12. The method of claim 8 wherein the modulator is a positive allosteric modulator of GPR4.

13. The method of claim 8 in which the neuronal cell is maintained in a cell culture.

14. The method of claim 8 in which the neuronal cell is located in an organoid.

15. The method of claim 8 in which the neuronal cell is human.

16. The method of claim 15 in which the human neuronal cell is located in the brain of an individual with Alzheimer's disease or Mild Cognitive Impairment.

17. A method of diagnosing a subject heterozygous or homozygous for the APOE4 allele at risk of having Alzheimer's disease or Mild Cognitive Impairment comprising:
determining the level of expression or activity of an expression product of GPR4, and;
determining the level of an expression product of at least one gene selected from YAP1, CTGF, CCND3, BDNF, ITGB3 and VCL in a tissue, cell or body fluid of said subject, and;
wherein for GPR4 the gene expression or activity is decreased, and;
for at least one of YAP1, CTGF, CCND3, BDNF and VCL a decrease, or for ITGB3 an increase, in the level of the expression product of the gene indicates elevated risk of Alzheimer's disease or Mild Cognitive impairment.

18. The method of claim 17 wherein the subject is a human.

19. The method of claim 18 wherein the subject is homozygous for the APOE4 allele.

20. The method of claim 18 wherein the cell is a human lymphocyte.

21. A method for selecting a therapeutic modulator of GPR4 for administration to a subject having, or at-risk of having, Alzheimer's disease or Mild Cognitive Impairment, said method comprising:
measuring the level of expression of a gene selected from YAP1, CTGF, CCND3, BDNF, ITGB3 and VCL in a biological sample from the subject, and;
selecting said therapeutic modulator based on whether the subject demonstrates a decrease of the expression product thereof for YAP1, CTGF, CCND3, BDNF or VCL or an increase of the expression product for ITGB3 in said sample.

22. A screening assay for identifying a modulator of the expression of a gene or protein selected from among YAP1, CTGF, CCND3, BDNF, VCL and ITGB3 comprising:
providing cells having an APOE4 allele, and
measuring the expression of at least one gene or protein selected from YAP1, CTGF, CCND3, BDNF, VCL and ITGB3, and
exposing the cells to modulators that are modulators of GPR4 activity, and
measuring the expression of at least one gene or protein selected from YAP1, CTGF, CCND3, BDNF, VCL and ITGB3 in the cells after the exposure, and
identifying modulators that alter the expression of a gene or protein of at least one of YAP1, CTGF, CCND3, BDNF, VCL and ITGB3.

23. The method of claim 22 wherein the modulator is a small molecule.

24. The method of claim 22 in which the cell is a neuronal cell.

25. The method of claim 22 in which the cell is a lymphocyte.

26. The method of claim 22 in which the cell is maintained in a cell culture.

27. The method of claim 22 in which the cell is homozygous for the APOE4 allele.

28. The method of claim 22 in which the neuronal cell is located in an organoid.

29. The method of claim 22 in which the cell is human.

30. The method of claim 22 in which the cell is murine.

31. The method of claim 22 in which the modulator increases the expression of at least one gene selected from YAP1, CTGF, CCND3, BDNF and VCL.

32. The method of claim 22 in which the modulator decreases the expression of ITGB3.

33. The method of claim 22 in which the human neuronal cell is located in the brain of an individual with Alzheimer's disease or Mild Cognitive Impairment

34. The method of claim 22 where the screen is a high-throughput screen.
